(19) United States
(12) Patent Application Publication
(10) Pub. No.: US 2024/0398732 A1
(43) Pub. Date: Dec. 5, 2024

(54) Treating Cancers with Combinations of Spironolactone and Acylfulvenes

(71) Applicant: LANTERN PHARMA INC., Dallas, TX (US)

(72) Inventors: Jianli Zhou, Dallas, TX (US); Aditya Kulkarni, Dallas, TX (US); Kishor Bhatia, Ellicott, MD (US)

(73) Assignee: LANTERN PHARMA INC., Dallas, TX (US)

(21) Appl. No.: 18/425,632

(22) Filed: Jan. 29, 2024

Related U.S. Application Data
(63) Continuation of application No. PCT/US2022/074297, filed on Jul. 29, 2022.
(60) Provisional application No. 63/227,257, filed on Jul. 29, 2021.

(51) Int. Cl. A61K 31/17; A61K 31/585; A61K 45/06; A61P 35/00

(52) U.S. Cl. CPC A61K 31/17; A61K 31/585; A61K 45/06; A61P 35/00

(57) ABSTRACT

A method of treating cancer includes a combination of a therapeutically effective amount of an illudin or an illudin analog thereof, derivative, or a pharmaceutically acceptable salt thereof, and a therapeutically effective amount of a spironolactone or an analog, derivative, or a pharmaceutically acceptable salt thereof. Compositions and kits of the same are included herein.

### TECHNICAL FIELD

This application relates to cancer treatments and more specifically this application relates to cancer treatments using a combination therapy including a spironolactone.

### BACKGROUND

Cancer is one of the most common causes of death in people. The development of therapeutic strategies for patients with advanced cancer has markedly improved overall survival. However, resistance to anticancer reagents is inevitable, and the prognosis of advanced cancer remains poor. There are several potential sources of cancer drug resistance, including alterations to drug transporters, the suppression of apoptosis, mitochondrial alterations, the promotion of DNA damage repair, autophagy, epithelial-mesenchymal transition, and cancer stem cells (CSCs). Appropriate strategies that consider the mechanisms are necessary to cure cancer.

Combination-therapy treatments for cancer have become more common, in part due to the perceived advantage of attacking the disease via multiple avenues. Although many effective combination-therapy treatments have been identified over the past few decades; in view of the continuing high number of deaths each year resulting from cancer, a continuing need exists to identify effective therapeutic regimens for use in anticancer treatment.

Accordingly, there is always a need for improved methods to treat cancer.

### SUMMARY

This application discloses the discovery that treatment of a cancer in a subject with a combination of an illudin or illudin analog (e.g., acylfulvene) and a spironolactone has synergistically greater effects (i.e., greater than the effects of each added together) than the effects provided by either acylfulvene or spironolactone treatment, alone. For example, the cytotoxicity delivered from treating a cancer with a combination of illudin or acylfulvene and a spironolactone is unexpectedly greater compared to the cytotoxicity delivered when treating the cancer with illudin or acylfulvene or spironolactone, alone (or greater than the cytotoxicity of both added together). In addition, it was discovered that the combination therapy results in more rapid killing of cancer cells and more rapid tumor shrinkage than was found when either therapy, alone, was used.

One aspect of this application includes a combination therapy for treating cancers. In embodiments, the therapy includes administering a combination of active agents including an illudin or illudin analog (e.g., acylfulvene), and a spironolactone.

Another aspect of this application provides pharmaceutical compositions comprising an illudin or illudin analog (e.g., acylfulvene) and a spironolactone or pharmaceutically acceptable salts thereof, mixed with pharmaceutically suitable carriers or excipient(s) at doses to treat or prevent cancer. The pharmaceutical compositions can also be administered in combination with other therapeutic agents or therapeutic modalities simultaneously, sequentially, or in alternation.

Another aspect of this application includes therapeutically effective amounts of each illudin or acylfulvene, and a spironolactone used in combination that will be lower when used in combination in comparison to monotherapy with each agent alone. Such lower therapeutically effective amount could afford for lower toxicity of the therapeutic regimen.

Another aspect of this application includes the therapy including an acylfulvene that is (+)-hydroxyureamethyl acylfulvene.

Another aspect of this application includes the therapy including an acylfulvene that is Irofulven.

Another aspect of this application includes the treatments of cancer that can include solid tumors, and hematological malignances. Tumors such as, but not limited to, hyperplastic or neoplastic disease, such as a carcinoma, sarcoma, or mixed type cancer, including breast, colon, rectal, endometrial, gastric, prostate or brain, mesothelioma, ovarian, lung or pancreatic cancer can be targeted for therapy.

### BRIEF DESCRIPTION OF THE FIGURES

FIG. 1A shows cell growth rates in U87 glioblastoma cells with an exemplary treatment including LP-184;

FIG. 1B shows cell growth rates in CHLA-06 atypical teratoid rhabdoid tumor cells with an exemplary treatment including LP-184;

FIG. 1C shows cell growth rates in CAKI-2 papillary renal cell carcinoma cell lines with an exemplary treatment including LP-184;

FIG. 2A shows cell growth in RPMI 8226 human myeloma cell lines with an exemplary treatment including LP-284;

FIG. 2B shows cell growth in SUDHL6 cell lines with an exemplary treatment including LP-284;

FIG. 3 shows treatment of GBM cells with LP-184 and spironolactone;

FIG. 4A shows cell growth rates in M1123 glioblastoma cells with an exemplary treatment including LP-184;

FIG. 4B shows cell growth rates in Mayo39 GBM neurosphere cells with an exemplary treatment including LP-184;

FIG. 4B shows cell growth rates in U87 glioblastoma cells with an exemplary treatment including LP-184;

FIG. 5 shows cell grow rates with lower doses of LP-184 and spironolactone in M1123 cell line.

FIG. 6 shows cell grow rates with lower doses of LP-184 and spironolactone in Mayo39 cell line.

FIG. 7 shows cell grow rates with lower doses of LP-184 and spironolactone in U87 cell line.

FIG. 8 shows synergy of LP-184 and spironolactone.

FIG. 9A shows cell growth rates in ARTR atypical teratoid rhabdoid tumor cells with an exemplary treatment including LP-100

FIG. 9B shows cell growth rates in 22RVI prostate cancer cells with an exemplary treatment including LP-100

FIG. 9C shows cell growth rates in ACHN renal cancer cells with an exemplary treatment including LP-100

FIG. 10 shows that spironolactone enhanced the effect at lower doses of LP-100 in MDAPCA2b cell lines.

### DETAILED DESCRIPTION

This application provides a combination therapy for treating solid cancers and blood cancers. In embodiments, the therapy includes administering a combination of active agents including an illudin or an illudin analog (e.g., acylfulvene) and a spironolactone. In other embodiments, the therapy includes administering a combination of other therapies. In other embodiments, the combination therapy can be used to treat biochemical occurrence or recurrence of solid cancers (e.g., lung cancer, breast cancer, prostate cancer, colon cancer, rectum cancer, and bladder cancer), glioblastoma and atypical teratoid rhabdoid, and renal cell carcinoma). In other embodiments, the therapy includes the combination therapy can be used to treat biochemical occurrence and recurrence of blood cancers in which an acylfulvene (e.g., hydroxyureamethyl acylfulvene) or salt thereof and a spironolactone administered in a therapeutically effective amount to the patient. In certain embodiments, the combination can provide a treatment for lymphoma, such as mantle cell lymphoma (MCL) and double-hit lymphoma (DHL). In multiple myeloma (MM), the overgrowth of plasma cells in the bone marrow can crowd out normal blood-forming cells.

### Illudin or Acylfulvene

In one embodiment, this application includes the use of an illudin or illudin analog (e.g., acylfulvene). Acylfulvene is a class of cytotoxic semi-synthetic derivatives of illudin, a natural product that can be extracted from the jack o′lantern mushroom (Omphalotus olearius). Acylfulvene, derived from the sesquiterpene illudin S by treatment with acid (reverse Prins reaction), is far less reactive to thiols than illudin S.

In one example, the acylfulvene is (−)-hydroxyureamethyl acylfulvene (termed LP-184 by Lantern Pharma Inc.), which shifts light positively, is shown below:

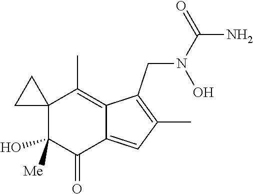 [SMILES: CC1=C(CN(O)C(N)=O)C2=C(C)C3(CC3)[C@@](C)(O)C(=O)C2=C1]

In another example, the acylfulvene is (+)-hydroxyureamethyl acylfulvene (termed LP-284 by Lantern Pharma Inc.), which shifts light negatively, is shown below:

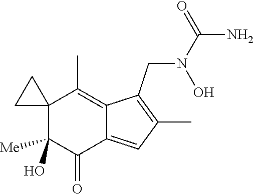 [SMILES: CC1=C(CN(O)C(N)=O)C2=C(C)C3(CC3)[C@](C)(O)C(=O)C2=C1]

(+)-hydroxyureamethyl acylfulvene and (−)-hydroxyureamethyl acylfulvene are enantiomers and are now known publicly.

In another example, the acylfulvene is Irofulven.

### Spironolactone

Chemical names for spironolactone include 7α-Acetylthiospirolactone, 7α-Acetylthio-17α-hydroxy-3-oxopregn-4-ene-21-carboxylic acid γ-lactone, 7α-Acetylthio-3-oxo-17α-pregn-4-ene-21,17.beta.-carbolactone, 3-(3-Oxo-7α-acetylthio-17.beta.-hydroxyandrost-4-en-17α-yl)pr- opionic acid lactone, 7α-Acetylthio-17α-(2-carboxyethyl)androst-4-en-17β-ol-3- one γ-lactone, and 7α-Acetylthio-17α-(2-carboxyethyl)testosterone γ-lactone). Other names include SC-9420 and NSC-150339.

Examples of analogs and derivatives of spironolactone include those that are closely related structurally such as canrenone, potassium canrenoate, drospirenone, and eplerenone, which are used clinically. Other examples include never-marketed spirolactones SC-5233 (6,7-dihydrocanrenone; 7a-desthioacetylspironolactone), SC-8109 (19-nor-6,7-dihydrocanrenone), spiroxasone, prorenone (SC-23133), mexrenone (SC-25152, ZK-32055), dicirenone (SC-26304), spirorenone (ZK-35973), and mespirenone (ZK-94679).

Spironolactone is a steroidal lactone compound having a significant aldosterone antagonistic character (U.S. Pat. No. 3,257,390). A spironolactone can include its derivatives, isomers, salts, and solvates thereof. Spironolactone is commercially available and has the molecular formula C₂₄H₃₂O₄S and a molar mass of 416.574 g mol-1.

Spironolactone has the following structure:

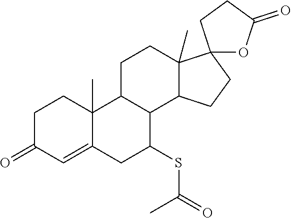 [SMILES: CC(=O)SC1CC2=CC(=O)CCC2(C)C2CCC3(C)C(CCC34CCC(=O)O4)C12]

In one embodiment, acylfulvene or hydroxyureamethyl acylfulvene or its salt may be administered either prior to, concomitantly with, or subsequent to the administration of a spironolactone.

One aspect of this application includes a method of treating cancer in a subject in need thereof. The method involves administering to the subject an effective amount of spironolactone and an effective amount of an acylfulvene. Spironolactone may be administered prior to or concomitantly with an acylfulvene for optimal synergistic effects. Spironolactone degrades the key Nucleotide Excision Repair (NER) protein XPB/ERCC3 (reference: https://www.ncbi.nlm.nih.gov/pmc/articles/PMC7277409/) and leads to NER deficiency. Subjects with NER deficiency are more sensitive to an illudin-based anti-cancer agent.

Another embodiment includes a pharmaceutical composition having a therapeutically effective amount of an illudin or an illudin analog thereof, derivative, or a pharmaceutically acceptable salt thereof, and a therapeutically effective amount of a spironolactone or an analog, derivative, or a pharmaceutically acceptable salt thereof. The illudin analog can be HydroxyUreaMethyl Acylfulvene.

In another embodiment, a kit for the treatment of cancer in a subject includes a therapeutically effective amount of an illudin or an illudin analog thereof, derivative, or a pharmaceutically acceptable salt thereof; and a therapeutically effective amount of a spironolactone or an analog, derivative, or a pharmaceutically acceptable salt thereof:

In another embodiment, the second therapeutic is one or more chemotherapeutic agents selected from camptothecin derivatives, paclitaxel, docetaxel, epothilone B, 5-FU, gemcitabine, oxaliplatin, cisplatinum, carboplatin, melphalam, dacarbazine, temozolomide, doxorubicin, imatinib, erlotinib, bevacizumab, cetuximab and a Raf kinase inhibitor.

In another embodiment, the second therapeutic is one or more chemotherapeutic agents selected from paclitaxel or cisplatinum.

The term “combination therapy” can include or includes the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In another aspect, a composition or combination therapy herein, or a pharmaceutically acceptable salt or solvate thereof, may be administered in combination with radiation therapy. Radiation therapy can also be administered in combination with a composition of the present invention and another chemotherapeutic agent described herein as part of a multiple agent therapy.

Combination therapy can be achieved by administering two or more agents, e.g., an acylfulvene, a spironolactone and one or more other therapeutic agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present within the patient's body at the same time, this need not be so.

The methods of combination therapy may or should result in a synergistic effect, wherein the effect of a combination of compounds or other therapeutic agents is greater than the sum of the effects resulting from administration of any of the compounds or other therapeutic agents as single agents. A synergistic effect may also be an effect that cannot be achieved by administration of any of the compounds or other therapeutic agents as single agents. The synergistic effect may include, but is not limited to, an effect of treating cancer by reducing tumor size, inhibiting tumor growth, or increasing survival of the subject The synergistic effect may also include reducing cancer cell viability, inducing cancer cell death, and inhibiting or delaying cancer cell growth.

Therapeutically effective doses can vary, as recognized by those skilled in the art, depending on the diseases treated, the severity of the disease, the route of administration, the age and general health condition of the patient, excipient usage, the possibility of co-usage with other therapeutic treatments such as use of other agents and the judgment of the treating physician. For example, guidance for selecting an effective dose can be determined by reference to the prescribing information for hydroxyureamethyl acylfulvene or journal discussion the same.

The term “effective amount” as used herein refers to the amount of an agent needed to alleviate at least one or more symptoms of the disease or disorder, and relates to a sufficient amount of pharmacological composition to provide the desired effect. The term “therapeutically effective amount” therefore refers to an amount of the agent that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

The dosage ranges for the administration of an agent according to the methods described herein depend upon, for example, the form of the agent, its potency, and the extent to which symptoms, markers, or indicators of a condition described herein are desired to be reduced, for example, the percentage reduction desired for tumor growth. The dosage should not be so large as to cause adverse side effects. Generally, the dosage will vary with the age, condition, and sex of the patient and can be determined by one of skill in the art. The dosage can also be adjusted by the individual physician in the event of any complication.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder.

The efficacy of an agent described herein in, e.g., the treatment of a condition described herein, or to induce a response as described herein (e.g., solid cancers or blood cancers) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. tumor size and/or growth rate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example, treatment of blood cancers in a mouse model. When using an experimental animal model, efficacy of treatment is evidenced when a statistically significant change in a marker is observed, e.g. tumor size and/or growth rate. In some embodiments, the therapeutically effective amount of hydroxyureamethyl-acylfulvene or a pharmaceutically acceptable salt thereof is selected from the group consisting of 0.5 mg/day, 1 mg/day, 2.5 mg/day, 5 mg/day, 10 mg/day, 20 mg/day, 30 mg/day, 60 mg/day, 90 mg/day, 120 mg/day, 150 mg/day, 180 mg/day, 210 mg/day, 240 mg/day, 270 mg/day, 300 mg/day, 360 mg/day, 400 mg/day, 440 mg/day, 480 mg/day, 520 mg/day 580 mg/day, 600 mg/day, 620 mg/day, 640 mg/day, 680 mg/day, and 720 mg/day.

The administration dose should be adjusted for the requirement of the individual in need. It is known that in humans the administration of 25 to 50 mg of spironolactone daily for the treatment of cardiac failure and from 100 to 400 mg daily for the treatment of hyperaldosteronism. The use of spironolactone in the range from 25 to 400 mg has been studied regarding the potential adverse effect. For the treatment of cancer, the dose can range from 25 to 400 mg daily. For illustration, spironolactone dose concentrations that can be used range from 5-25 μM, 25-100 mg/kg in mice (i.p.), and 20-200 mg daily (oral) clinically in humans.

The term “treat” is used and includes both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

The pharmaceutical compositions can be included in a container, pack, or dispenser together with instructions for administration.

The composition of the present invention is capable of further forming salts. The composition of the present invention can form more than one salt per molecule, e.g., mono-, di-, tri-All of these forms are also contemplated within the scope of the claimed invention.

As used herein, “pharmaceutically acceptable salts” refer to derivatives of the compounds of the present invention wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamie, sulfanilie, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl) benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present invention also encompasses salts formed when an acidic proton in the parent compound either is replaced by a metal ion, e.g., an alkali metal ion, an alkaline earth ion, or an aluminum ion, or coordinates with an organic base such as ethanolamine, diethanolamine, triethanolamine, tromethamine, N-methylglucamine, and the like.

It should be understood that all references to pharmaceutically acceptable salts include solvent addition forms (solvates), of the same salt.

As used herein, the term “selectively” means tending to occur at a higher frequency in one population than in another population. The compared populations can be cell populations. Preferably, a compound of the present invention, or a pharmaceutically acceptable salt or solvate thereof, acts selectively on a cancer or precancerous cell but not on a normal cell. Preferably, a compound of the present invention, or a pharmaceutically acceptable salt or solvate thereof, acts selectively to modulate one molecular target (e.g., nucleotide excision repair (NER) players ERCC3). The invention also provides a method for selectively inhibiting the activity of an enzyme, such as a NER proteins. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

The composition, or pharmaceutically acceptable salts or solvates thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the invention can be found in Remington: the Science and Practice of Pharmacy, 19.sup.th edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

As used herein, a “subject in need thereof” is a subject having a precancerous condition. Preferably, a subject in need thereof has cancer A “subject” includes a mammal. The mammal can be e.g., any mammal, e.g., a human, primate, bird, mouse, rat, dog, cat, cow, horse, goat, camel, sheep or a pig. Preferably, the mammal is a human. The subject of the present invention includes any human subject who has been diagnosed with, has symptoms of, or is at risk of developing a cancer or a precancerous condition.

A subject in need thereof may have refractory or resistant cancer. “Refractory or resistant cancer” means cancer that does not respond to treatment. The cancer may be resistant at the beginning of treatment or it may become resistant during treatment. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy. In certain embodiments the prior therapy is monotherapy. In certain embodiments the prior therapy is combination therapy.

In some embodiments, a subject in need thereof may have a secondary cancer as a result of a previous therapy. “Secondary cancer” means cancer that arises due to or as a result from previous carcinogenic therapies, such as chemotherapy.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater, even more preferably, reduced by 50% or greater; and most preferably, reduced by greater than 75% or greater. Size of a tumor may be measured by any reproducible means of measurement. The size of a tumor may be measured as a diameter of the tumor.

Treating cancer results in a decrease in number and size of tumors. Preferably, after treatment, tumor number or size is reduced by 5% or greater relative to number prior to treatment; more preferably, tumor number or size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2x, 3x, 4x, 5x, 10x, or 50x.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater: even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2x, 3x, 4x, 5x, 10x, or 50x.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt or solvate thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt or solvate thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. After treatment, tumor regrowth can be less than 5%, more preferably, tumor regrowth can be less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%, and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%, more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

Administering a composition of the present invention to a cell or a subject in need thereof can result in modulation (i.e., stimulation or inhibition) of an activity of a protein methyltransferase of interest

Treating cancer or a cell proliferative disorder can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. Several cells in a population can be measured by fluorescence activated cell sorting (FACS), immunofluorescence microscopy and light microscopy. Methods of measuring cell death are as shown in Li et al., Proc. Natl. Acad. Sci. USA. 100(5): 2674-8, 2003. In an aspect, cell death occurs by apoptosis.

Preferably, an effective amount of a composition of the present invention, or a pharmaceutically acceptable salt or solvate thereof, is not significantly cytotoxic to normal cells. A therapeutically effective amount of a compound is not significantly cytotoxic to normal cells if administration of the compound in a therapeutically effective amount does not induce cell death in greater than 10% of normal cells. A therapeutically effective amount of a compound does not significantly affect the viability of normal cells if administration of the compound in a therapeutically effective amount does not induce cell death in greater than 10% of normal cells. In an aspect, cell death occurs by apoptosis.

Contacting a cell with a composition of the present invention, or a pharmaceutically acceptable salt or solvate thereof, can induce, or activate cell death selectively in cancer cells. Administering to a subject in need thereof a compound of the present invention, or a pharmaceutically acceptable salt or solvate thereof, can induce or activate cell death selectively in cancer cells. Contacting a cell with a composition of the present invention, or a pharmaceutically acceptable salt or solvate thereof, can induce cell death selectively in one or more cells affected by a cell proliferative disorder. Preferably, administering to a subject in need thereof a composition of the present invention, or a pharmaceutically acceptable salt or solvate thereof, induces cell death selectively in one or more cells affected by a cell proliferative disorder.

This application relates to a method of treating or preventing cancer by administering a composition of the present invention, or a pharmaceutically acceptable salt or solvate thereof, to a subject in need thereof, where administration of the composition of the present invention, or a pharmaceutically acceptable salt or solvate thereof, results in one or more of the following: prevention of cancer cell proliferation by accumulation of cells in one or more phases of the cell cycle (e.g. G1, G1/S, G2/M), or induction of cell senescence, or promotion of tumor cell differentiation; promotion of cell death in cancer cells via cytotoxicity, necrosis or apoptosis, without a significant amount of cell death in normal cells, antitumor activity in animals with a therapeutic index of at least 2. As used herein, “therapeutic index” is the maximum tolerated dose divided by the efficacious dose.

The term “kit” means a combination partners as defined above can be dosed independently or by use of different fixed combinations with distinguished amounts of the combination partners, i.e., simultaneously or at different time points. The parts of the kit of parts can then, e.g., be administered simultaneously or chronologically staggered, that is at different time points and with equal or different time intervals for any part of the kit of parts. The ratio of the total amounts of the combination partners to be administered in the combined preparation can be varied. The combination partners can be administered by the same route or by different routes.

One skilled in the art may refer to general reference texts for detailed descriptions of known techniques discussed herein or equivalent techniques. These texts can, of course, also be referred to in making or using an aspect of the invention.

### EXAMPLES

In order that the disclosure disclosed herein may be more efficiently understood, examples are provided below. It should be understood that these examples are for illustrative purposes only and are not to be construed as limiting the disclosure in any manner.

LP-100 (Irofulven), LP-184 and LP-284 belong to the acylfulvene compound family known to induce DNA lesions repaired by the Transcription-Coupled Nucleotide Excision Repair (TC-NER) pathway. If the TC-NER pathway is impaired, DNA damage can no longer be repaired, and cell death will occur.

Spironolactone selectively induces cell death and inhibits the growth of cancer cells. As shown below, as little as 10 μM Spironolactone alone did not cause reduced cell survival. In combination with an acylfulvene, the cells or tumors were killed or reduced.

### Example 1

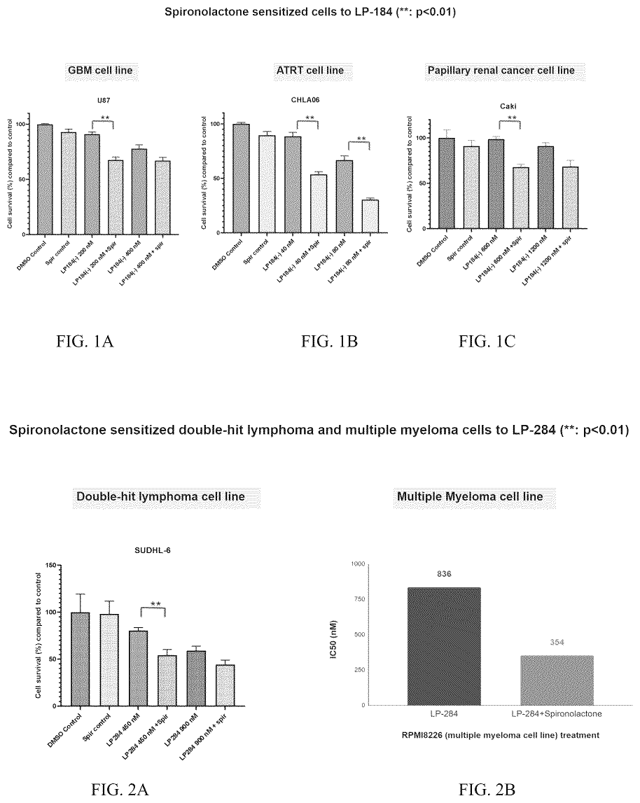

FIGS. 1A, 1B, and IC show that combining 10 μM spironolactone with LP-184 in treating U87 (glioblastoma cell line), CHLA-06 (atypical teratoid rhabdoid tumor cell line), and CAKI-2 (papillary renal cell carcinoma cell line) resulted in significantly lower percentage of cell survival than using LP-184 alone. As can be seen, spironolactone alone did not substantially change the growth rates.

### Example 2

FIG. 2A and FIG. 2B shows that while a 10 μM spironolactone treatment alone did not cause cytotoxicity to the multiple myeloma cell line RPMI 8226 human myeloma cell lines, it reduced LP-284 IC50 by 2.4 fold. Additionally, the percentage of cell survival was significantly reduced when the SUDHL6 double-hit lymphoma cell line was treated with 450 nM LP-284 plus 10 uM spironolactone compared with 450 nM LP-284 alone.

### Example 3

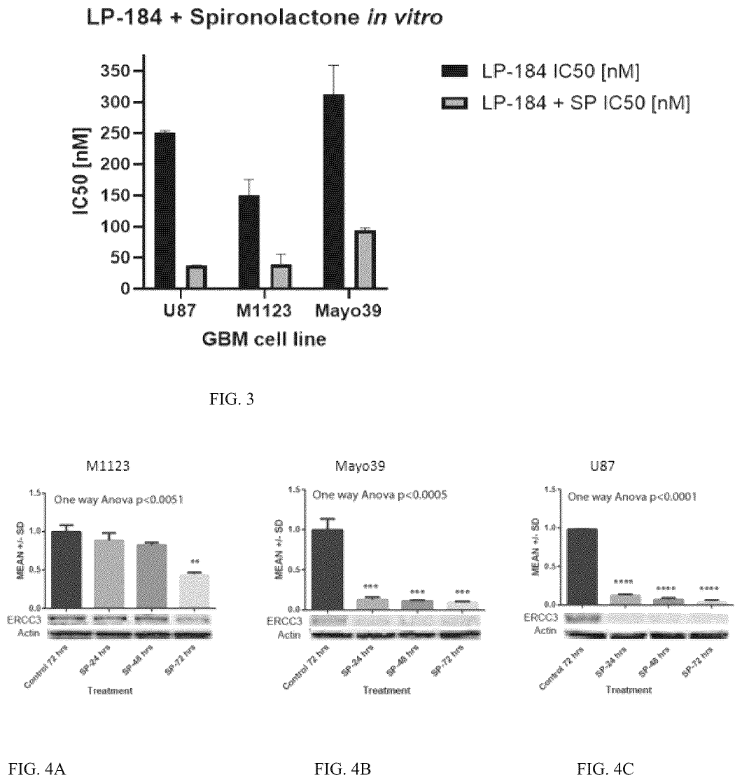

FIG. 3 shows that treatment of GBM cells with LP-184 and 5 μM Spironolactone resulted in a 3-6 fold decrease in LP-184 IC50s in U87, M1123 and Mayo39 glioblastoma cultures in vitro. FIG. 3 shows relative IC50 data/cell viablity with standard error and Table 1 lists the mean IC50 values.

TABLE 1

| | LP-184 alone | LP-184 IC50 [mean value, nM] |
|---|---|---|
| Glioblastoma | IC50 [mean | in combination with 5 uM |
| cell line | value, nM] | Spironolactone |
| U87 | 250.3 | 37.6 |
| M1123 | 151.1 | 39.7 |
| Mayo39 | 312.8 | 94.3 |

### Example 3

FIGS. 4A, 4B, and 4C show that a 10 μM spironolactone treatment to M1123 (glioblastoma cell line), Mayo39 (Glioblastoma cell line), and U87 (glioblastoma cell line) results in a time-dependent depletion of the TC-NER component ERCC3 at the protein level as measured by western blot analysis. Representative images show quantification of ERCC3 western blot intensity with significant Spironolactone-mediated ERCC3 downregulation over 24-72 hours in different glioblastoma cell lines.

### Example 4

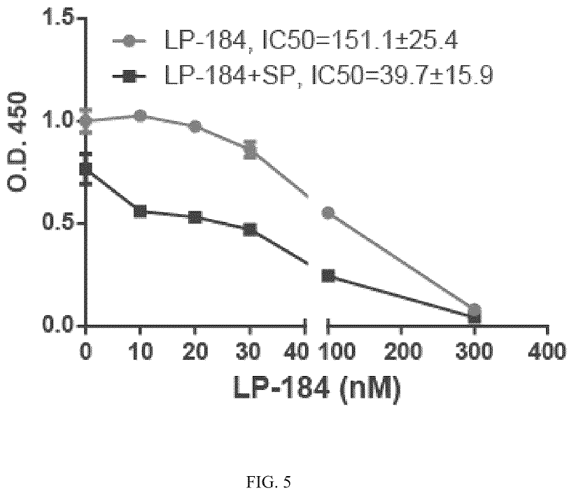

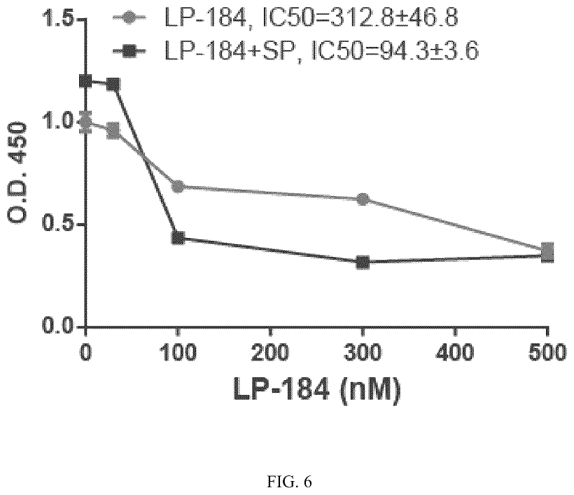

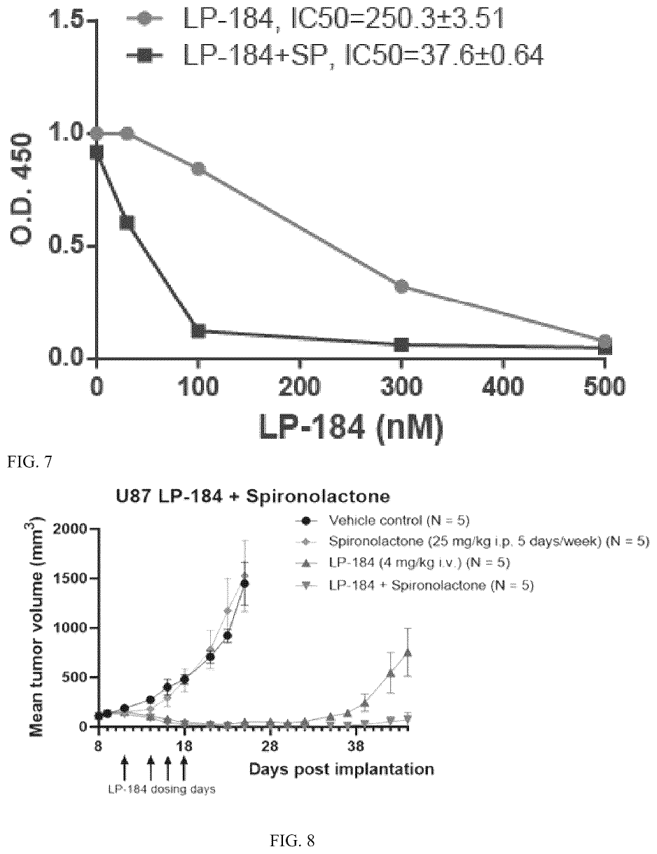

FIG. 5, FIG. 6, and FIG. 7, respectively, show that spironolactone enhanced the effect at lower doses of LP-184 in M1123, Mayo39 and U87 cell lines. This shows that therapeutically effective amounts of LP-184 with a spironolactone is lower than when LP-184 is used in as a monotherapy alone.

### Example 5

FIG. 8 shows synergy of LP-184 and spironolactone. SCID mice bearing pre-established subcutaneous U87 xenograft tumors were treated with Spironolactone alone, LP-184 alone or their combination. Spironolactone treatment began on post implantation day 8 at 25 mg/kg intraperitoneally 5 days per week, and LP-184 on day 9 at 4 mg/kg intravenously every other day for 4 doses. Spironolactone monotherapy had no effect on tumor growth compared to untreated controls. As shown in FIG. 2, LP-184 alone and combined with Spironolactone induced complete or near complete tumor regression. Beginning on ˜day 25,tumor growth re-emerged in 5/5 animals treated with LP-184 alone vs tumor regrowth in only 1/5 animals treated with LP-184+Spironolactone (persistent complete response in 4/5 tumors). Tumor volume was significantly reduced in LP-184 and Spironolactone versus LP-184 alone beginning on day 8, and the magnitude of mean differences continued to increase up to the final day of observation on day 42 where mean volume was 10-fold lower in LP-184+Spironolactone treated mice (p value 0.048).

### Example 6

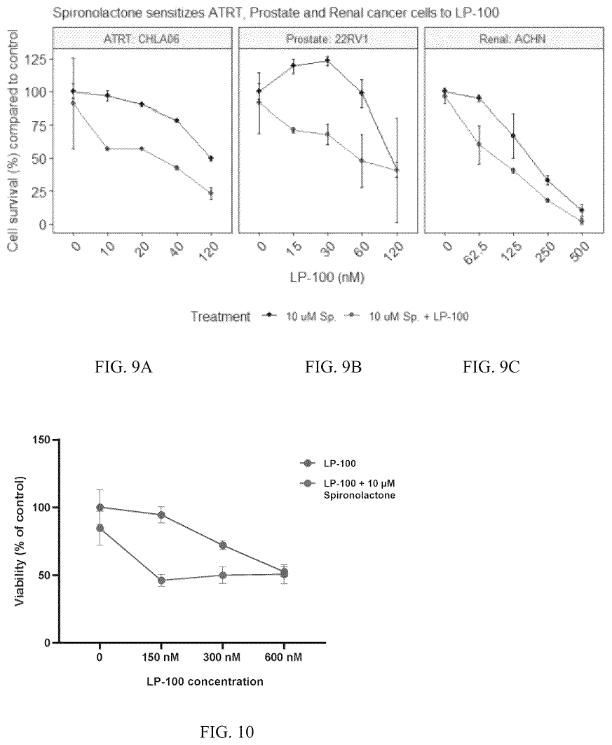

FIGS. 9A, 9B, and 9C show that adding 10 μM spironolactone to CHLA 06 (atypical teratoid rhabdoid tumor cell line), 22RV1 (prostate cell line), and ACHN (renal cell carcinoma cell line) did not change the cell growth rates. Further, the cell viability data shows that combining 10 uM spironolactone with LP-100 in cell treatment resulted in significantly lower percentage of cell survival than using LP-100 alone.

### Example 7

FIG. 10 shows that spironolactone enhanced the effect at lower doses of LP-100 in MDAPCA2b cell lines. Using combination of Irofulven with spironolactone (or other agents that inhibit the function of DNA repair proteins involved in the TC-NER pathway) to potentiate tumor cell killing.

### Example 8—Synergy Score

MacSynergy II software was used to score the combination of LP-184 and spironolactone. This program allows the three-dimensional examination of drug interactions of all data points generated from the checkerboard combination of two inhibitors with Bliss-Independence model. Confidence bounds are determined from replicate data. If the 95% confidence limits (CL) do not overlap the theoretic additive surface, then the interaction between the two drugs differs significantly from additive. The volumes of synergy or antagonism can be determined and graphically depicted in three dimensions and represent the relative quantity of synergism or antagonism per change in the two drug concentrations. Synergy and antagonism volumes are based on the Bliss independence model, which assumes that both compounds act independently on different targets. A set of predicted fractional responses faAB under the Bliss independence model is calculated as faAB=faA+faB−faA faB with faA andfaß representing the fraction of possible responses, e.g. percent (%) inhibition, of compounds A and B at amounts dA and dB, respectively, and describes the percent inhibition of a combination of compounds A and B at amount (dA+dB). The 95% synergy/antagonism volumes are the summation of the differences between the observed inhibition and the 95% confidence limit on the prediction of faAB under the Bliss independence model. MacSynergy II was used for data analysis.

The combination of LP-184 and Spironolactone had a synergy volume between 14.10 and 15.52 μM (additive synergism). Bliss synergy scores were computed from in vitro pancreatic cancer cell viability data (Table 2). A score of >10 was considered indicative of synergy and <0 antagonism. Overall Bliss synergy scores of 14.08, 16.47 and 15.52 for LP-184 +spironolactone in Capan-1 (BRCA2 loss), Hs766t (ATR mutant) and Panc03.27 cell lines respectively were achieved. Spironolactone showed high synergy with LP-184 in all 3 cell lines tested. Table 2 shows those results in pancreatic cells.

TABLE 2
Spironolactone synergizes with LP-184
in vitro in pancreatic cancer cells

| Pancreatic | | Mean Overall Bliss Synergy Score |
|---|---|---|
| cancer | | for LP-184 + Spironolactone |
| cell line | DDR defect | combination in vitro |
| Capan-1 | BRCA2 loss | 14.08 ± 1.02 |
| Hs766t | ATR mutant | 16.47 ± 4.26 |
| Panc03.27 | None | 15.52 ± 0.73 |
| | characterized | |

While a number of exemplary aspects and embodiments have been discussed above, those of skill in the art will recognize certain modifications, permutations, additions, and sub-combinations thereof. It is therefore intended that the following appended claims and claims hereafter are interpreted to include all such modifications, permutations, additions, and sub-combinations as are within their true spirit and scope.

1. A method of treating cancer, the method comprises administering to a subject in need of treatment a combination of active agents comprising:
a. a therapeutically effective amount of hydroxyureamethylacylfulvene, or a pharmaceutically acceptable salt thereof; and
b. a therapeutically effective amount of a spironolactone or an analog, derivative, or a pharmaceutically acceptable salt thereof.

2. (canceled)

3. (canceled)

4. The method of claim 1, wherein the hydroxyureamethylacylfulvene has the following structure:

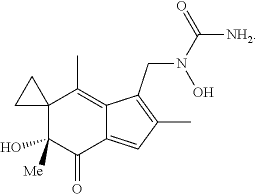 [SMILES: CC1=C(CN(O)C(N)=O)C2=C(C)C3(CC3)[C@@](C)(O)C(=O)C2=C1]

5. The method of claim 1, wherein the hydroxyureamethylacylfulvene has the following structure:

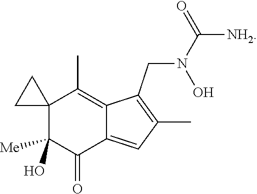 [SMILES: CC1=C(CN(O)C(N)=O)C2=C(C)C3(CC3)[C@](C)(O)C(=O)C2=C1]

6. (canceled).

7. The method of claim 1, wherein the hydroxyureamethylacylfulvene is administered prior to or concomitantly with the administration of spironolactonethe.

8. The method of claim 1, wherein the active agents are administered daily.

9. The method of claim 1, wherein the active agents are administered sequentially.

10. The method of claim 1, wherein the active agents are administered as a co-formulation.

11. The method of claim 1, wherein the hydroxyureamethylacylfulvene administration is after spironolactone administration.

12. The method of claim 1, the method further comprising administering radiotherapy, chemotherapy to, performing surgery on, the subject before, during, or following the illudin and/or administering the spironolactone.

13. The method of claim 1, wherein the cancer is a solid cancer selected from the group consisting of lung cancer, breast cancer, prostate cancer, colon cancer, rectum cancer, and bladder cancer.

14. The method of claims 1, wherein the subject is an animal.

15. The method of claim 1, wherein the subject or mammal is a human.

16. The method of claims 1, further comprising subjecting the subject to radiation therapy before, after, or during treatment with the HydroxyUreaMethylAcylfulvene.

17. The method of claim 1, further comprising administering an additional therapeutic agent selected from the group consisting of paclitaxel, cisplatinum, docetaxel, and gemcitabine.

18. The method of claim 1, wherein the cancer comprises a solid tumor.

19. The method of claim 18, wherein the solid tumor is a tumor of the breast, central nervous system, colon, skin, lung, ovary, prostate, pancreatic or kidney.

20. The method of claim 1, wherein the cancer is lymphoma, mantle cell lymphoma, double-hit lymphoma, and multiple myeloma.

21. A pharmaceutical composition comprising a therapeutically effective amount of of hydroxyureamethylacylfulvene and a therapeutically effective amount of spironolactone, wherein the composition is formulated for oral, intravenous, or intraperitoneal administration.

22. (canceled).

23. The pharmaceutical composition of claim 20, wherein the hydroxyureamethylacylfulvene has the following structure:

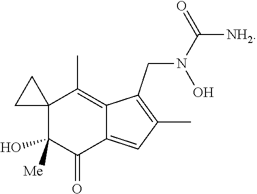 [SMILES: CC1=C(CN(O)C(N)=O)C2=C(C)C3(CC3)[C@@](C)(O)C(=O)C2=C1]

24. The pharmaceutical composition of claim 20, wherein the hydroxyureamethylacylfulvene has the following structure:

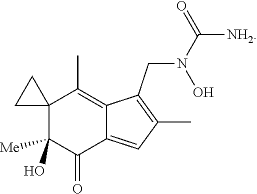 [SMILES: CC1=C(CN(O)C(N)=O)C2=C(C)C3(CC3)[C@](C)(O)C(=O)C2=C1]

25. (canceled).

26. A kit for the treatment of cancer in a subject comprising omprising a first container containing hydroxyureamethylacylfulvene and a second container containing spironolactone, with instructions for administering the combination therapy to the subject.

27. The method of claim 1, wherein the combination therapy results in a synergistic effect, reducing tumor size or inhibiting tumor growth more effectively than the administration of either hydroxyureamethylacylfulvene or spironolactone alone.

28. The method of claim 1, wherein the spironolactone selectively degrades the Nucleotide Excision Repair (NER) protein XPB/ERCC3 in cancer cells.

29. The method of claim 1, wherein the spironolactone is administered in combination with an agent that inhibits DNA repair proteins involved in the TC-NER pathway.

30. The method of claim 1, wherein the subject has refractory or resistant cancer that does not respond to prior treatments.

31. The method of claim 1, wherein the spironolactone is administered in a dosage ranging from 25 mg to 400 mg daily.
